(19) United States
(12) Patent Application Publication
(10) Pub. No.: US 2022/0347399 A1
(43) Pub. Date: Nov. 3, 2022

(54) SYSTEMS AND METHODS OF USING AN ULTRASONIC FOGGER TO DISTRIBUTE AN ANTISEPTIC SOLUTION

(71) Applicant: Wonder Spray, LLC, San Diego, CA (US)

(72) Inventor: John Burd, San Diego, CA (US)

(73) Assignee: Wonder Spray, LLC, San Diego, CA (US)

(21) Appl. No.: 17/724,425

(22) Filed: Apr. 19, 2022

Related U.S. Application Data
(60) Provisional application No. 63/182,668, filed on Apr. 30, 2021.

(51) Int. Cl. A61M 11/00; A61K 33/20

(52) U.S. Cl. CPC A61M 11/005; A61K 33/20; A61M 2202/02; A61M 2205/0294

(57) ABSTRACT

Antiseptic agent delivery systems and methods are provided, one such system comprising: a reservoir configured to store an antiseptic solution, wherein the antiseptic solution comprises an aqueous solution of hypochlorous acid; an ultrasound generator coupled to the reservoir, wherein the ultrasound generator vaporizes the antiseptic solution into an antiseptic vapor; a nozzle to direct the antiseptic vapor to an affected area; and a housing coupled to the reservoir and the nozzle, wherein the housing comprises a panel to control the ultrasound generator.

### CROSS-REFERENCE TO RELATED APPLICATIONS

The present application claims priority to U.S. Provisional Patent Application No. 63/182,668, filed Apr. 30, 2021, the content of which is incorporated herein in its entirety.

### TECHNICAL FIELD

The present disclosure is generally related to an ultrasonic fogger, and more specifically, embodiments of the present disclosure relate to the ultrasonic fogger being used to distribute an antiseptic solution, such as, for example, hypochlorous acid (HOCl).

### SUMMARY

Embodiments of the application are directed toward an antiseptic agent delivery system comprising: a reservoir configured to store an antiseptic solution, wherein the antiseptic solution comprises an aqueous solution of hypochlorous acid; an ultrasound generator coupled to the reservoir, wherein the ultrasound generator vaporizes the antiseptic solution into an antiseptic vapor; a nozzle to direct the antiseptic vapor to an affected area; and a housing coupled to the reservoir and the nozzle, wherein the housing comprises a panel to control the ultrasound generator.

Further embodiments of the application are directed toward a method of disinfecting an affected area, the method comprising: delivering an antiseptic solution to the affected area using an antiseptic agent delivery system, wherein the antiseptic agent delivery system comprises: a reservoir configured to store an antiseptic solution, wherein the antiseptic solution comprises an aqueous solution of hypochlorous acid; an ultrasound generator coupled to the reservoir, wherein the ultrasound generator vaporizes the antiseptic solution into an antiseptic vapor; a nozzle to direct the antiseptic vapor to an affected area; and a housing coupled to the reservoir and the nozzle, wherein the housing comprises a panel to control the ultrasound generator; and wherein the antiseptic solution comprises an aqueous solution of hypochlorous acid.

### BRIEF DESCRIPTION OF THE DRAWINGS

FIG. 1 illustrates an example of an antiseptic agent delivery system for use with HOCl, according to an implementation of the disclosure.

FIG. 2 illustrates a process of delivering HOCl using an antiseptic agent delivery system, according to an implementation of the disclosure.

FIG. 3 may illustrate an example antiseptic agent delivery system, according to an implementation of the disclosure.

FIG. 4 may illustrate an example antiseptic agent delivery system, according to an implementation of the disclosure.

FIG. 5 illustrates a computing or processing component capable of carrying out the functionality described herein.

### DETAILED DESCRIPTION

An ultrasonic fogger may be a device that uses ultrasonic energy to break water into individual water droplets or water vapor. These water vapor may appear to be a fog. In some embodiments, an ultrasonic fogger may include a shell or housing, a built-in AC/DC adapter and/or a battery, and/or an ultrasound generator. The fogger itself may be placed under one to four inches of a liquid and a built-in sensor may detect a presence of a liquid and activate the ultrasound generator. The ultrasound generator may vibrate rapidly at ultrasonic speeds and cause water molecules to break apart into individual droplets to form water vapor.

Hypochlorous acid (HOCl) is a weak acid that forms when sodium chloride dissolves in water, and itself partially dissociates, forming HOCl and hypochlorite, OCl-, depending on the solution pH. Similar to other chlorine-releasing agents (e.g., sodium hypochlorite, chlorine dioxide, and the N-chloro compounds such as sodium dichloroisocyanurate), aqueous HOCl is well known for its antimicrobial, anti-inflammatory, and immunomodulatory properties.

Applications of aqueous solutions containing approximately 10-2500 ppm (0.003% to 0.25%) HOCl are used in a variety of areas including (but not limited to) wound care, as antimicrobial agents, as anti-allergen agents, dental care and there are also significant applications in water treatments, food sanitization, and hard surface disinfection, and cosmetics.

HOCl is a potent antimicrobial capable of eradicating bacteria including antibiotic-resistant strains, viruses, fungi, and spores. In particular, HOCl is the active component responsible for pathogen disruption and inactivation by chlorine-releasing agents (CRAs). It is understood that the OCl-ion has little effect compared to undissolved HOCl, as the hypochlorite (OCl-), has only a minute effect compared to undissolved HOCl. Accordingly, the microbicidal effect of HOCl is the greatest when the percentage of undissolved HOCl is highest. In an aqueous solution of HOCl, ranging from approximately pH 4 to pH 7, chlorine exists predominantly as HOCl, whereas above pH 9, ClO-predominates.

Because HOCl is a highly active oxidizing agent, its mode of operation comprises destroying and/or deactivating cellular activity of proteins. For example, HOCl targets bacteria by chemically linking chlorine atoms to nucleotide bases that disrupt the function of bacterial DNA, impede metabolic pathways in which cells use enzymes to oxidize nutrients, and release energy, and other membrane-associated activities. Additionally, HOCl has also been found to disrupt oxidative phosphorylation and other membrane-associated activity. Similarly, HOCl has been found to inhibit bacterial growth. For example, at 50 mM (2.6 ppm), HOCl completely inhibited the growth of E. coli within 5 minutes, including inhibiting the DNA synthesis by ninety-six percent. Unlike conventional antibiotics, the antimicrobial activity of HOCl is directly toxic to microbial cells, including many Gram-positive and Gram-negative bacteria and their biofilms. HOCl has demonstrated disinfection efficacy against eradication of bacteria, including Acinetobacter baumannii, Bacillus subtilis, Enterobacter cloacae, Enterococcus faecalis, Escherichia coli, Escherichia coli, Escherichia coli, Enterobacter, Klebsiella pneumoniae, Listeria monocytogenes, MRSA (Staph. aureus), Polymicrobial biofilm, Proteus vulgaris, Pseudomonas aeruginosa, Salmonella choleraesuis, Shigella flexneri, Staph epidermidis, and Yersinia enterocolitica.

Additionally, HOCl possesses viricidal activity properties. For example, it has been demonstrated that HOCl inactivated naked f2 RNA at the same rate as RNA in intact phage, whereas f2 capsid proteins could still adsorb to the host. HOCl has demonstrated disinfection efficacy against eradication of viruses including norovirus, filoviruses such as Ebola, and human coronaviruses like MERS-CoV and SARS, as well as fungi such as Candida and Aspergillus. Further, as a sporicide, HOCl causes the spore coat to detach from the cortex, where further degradation occurs.

Both topical and internal applications of HOCl are safe because it is the exact same substance white blood cells in the human body produce to fight infection. Indeed, extensive studies have demonstrated exceptional safety of HOCl. The Food and Drug Administration (FDA) has cleared preparations of HOCl to be used, e.g., topically for wound cleansing, eye infections, tooth infections, nasal decontamination, and the care of surgical incisions. In particular, inhaling the vaporized form of HOCl has also been shown to causes no adverse effects.

The advent of antibiotics and other area disinfectants led to a reduction in environmental use of HOCl. However, widespread use of antibiotic agents led to antimicrobial resistance. Accordingly, an urgent need to optimize currently available anti-infectious therapies to overcome drug resistance exists. Antimicrobial resistance has not been observed for HOCl.

Embodiments of the technology disclosed herein are directed to antiseptic agent delivery systems in which the antiseptic agent is delivered to an affected area susceptible to infectious diseases caused by microbial (including spores), viral, fungal, allergy-causing agents via an ultrasonic fogger.

Some embodiments of the technology disclosed herein are directed to antiseptic agent delivery systems in which the antiseptic agent is administered, distributed, delivered, and/or otherwise applied via a pulmonary route as a treatment of infectious diseases caused by microbial (including spores), viral, fungal, allergy-causing agents, and/or other causes. Because the inhalation process gives a more direct access to the target organ/cavity than more traditional routes (e.g., topical, oral, intravenous, etc.), the pulmonary administration of HOCl used to inhibit bacterial growth provides a therapeutic approach that may help avoid and/or reduce antimicrobial resistance while alleviating the disease symptoms. For example, upper respiratory tract infections caused by one or more bacterial or viral pathogens such as bronchitis, epiglottitis, laryngitis, sinusitis, rhinosinusitis, chronic rhinosinusitis and so on, lung infections, such as pneumonia, may be treated by a pulmonary administration of an antiseptic agent, such as HOCl.

In some embodiments, a solution of HOCl may be delivered via the pulmonary route, administered, distributed, and/or otherwise applied via a fogger. In some implementations, the HOCl may be delivered, administered, distributed, and/or otherwise applied via a nebulizer, an aerosolizer, atomizer, and/or any other such delivery device. For example, a solution of HOCl of low concentration levels and relatively low acidotic pH may be used as a vaporized, nebulized, aerosolized, atomized topical laryngeal, tracheal, and alveolar disinfectant. In some embodiments, the aqueous solution of HOCl may include a concentration of at least approximately 0.01 percent of HOCl dissolved in water.

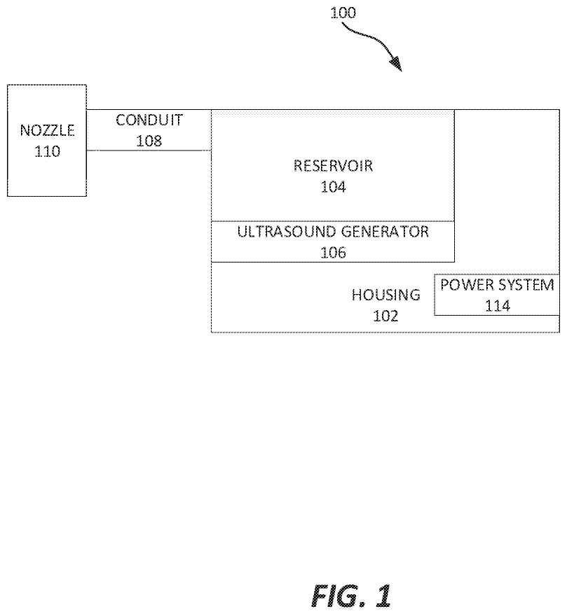

FIG. 1 depicts an antiseptic agent delivery system for delivering HOCl via the pulmonary route. The antiseptic agent delivery system 100 or components/features thereof may be implemented in combination with, or as an alternative to, other systems/features/components described herein, such as those described with reference to other embodiments and figures. The antiseptic agent delivery system 100 may additionally be utilized in any of the methods for using such systems/components/features described herein. The antiseptic agent delivery system 100 may also be used in various applications and/or permutations, which may or may not be noted in the illustrative embodiments described herein. For instance, antiseptic agent delivery system 100 may include more or fewer features/components than those shown in FIG. 1, in some embodiments. Moreover, the antiseptic agent delivery system 100 is not limited to the size, shape, number of components, etc. specifically shown in FIG. 1.

As shown in FIG. 1, the antiseptic agent delivery system 100 comprises a housing 102 which houses one or more components configured to vaporize the aqueous antiseptic solution so that it can be administered, distributed, delivered, and/or otherwise applied in the form of vapor by being inhaled into lungs, delivered, distributed, and/or otherwise applied to the affected area. For example, the one or more components hosed in housing 102 may include an ultrasound generator 106, transducer, or oscillator, a compressor, and/or similar components and associated circuitry (not shown) for causing vaporization. In some embodiments, the ultrasound generator 106 may comprise an electronic oscillator and one or more piezoelectric elements to create a vapor. The electronic oscillator may be configured to generate a high frequency ultrasonic wave, which causes the mechanical vibration of the one or more piezoelectric elements. The one or more piezoelectric elements may be in contact with a reservoir 102 used to store an aqueous HOCl solution. The one or more piezoelectric elements may vibrate at a high frequency and deliver a vapor comprising a vaporized HOCl. In some embodiments, these components may be controlled by a user using a control panel (not shown) to affect the resulting vapor size. For example, the vapor size may range from 0.5 microns to about 50 microns.

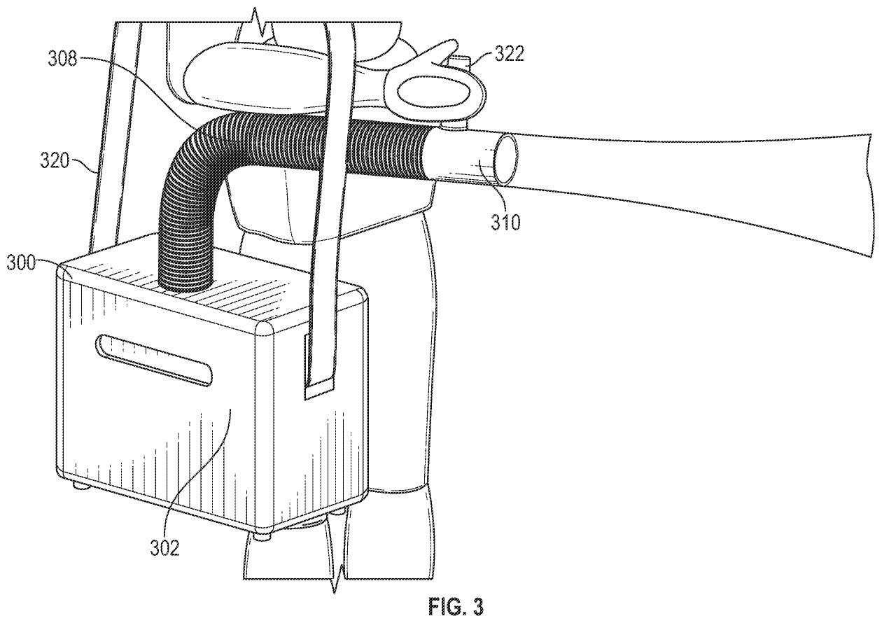

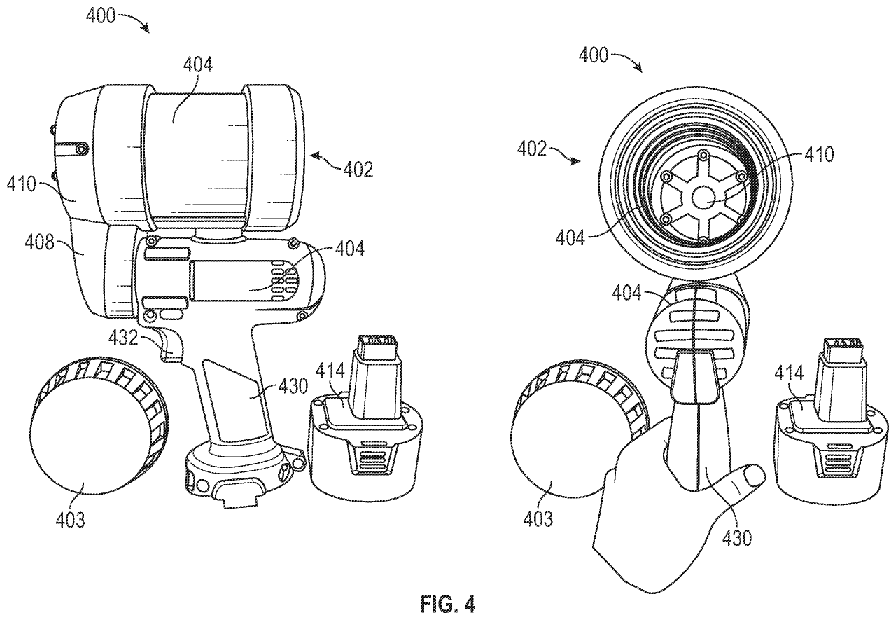

The antiseptic agent delivery system 100 may include a liquid supply reservoir 104 and a nozzle 110. In some embodiments, the aqueous antiseptic solution may be placed within the liquid supply reservoir 104. For example, the aqueous antiseptic solution may include liquid HOCl liquid solution ranging from 0.5 ml to 2 L placed in the reservoir 104. Nozzle 110 may be a single open tube, similar to the type shown in FIG. 3, while, in some embodiments, nozzle 110 may be closed and/or partially closed and multiple spray components may be used to administer, distribute, deliver, and/or otherwise apply the solution, as illustrated in FIG. 4. In some embodiments, nozzle 110 may be a mouthpiece for direct delivery to a patient. In embodiments, one or more nozzles may be used.

For example, FIG. 3 may illustrate an example antiseptic agent delivery system, in accordance with various embodiments of the present disclosure. As illustrated, the antiseptic agent delivery system 300 may be portable. The antiseptic agent delivery system 300 may include a strap 320 for holding the antiseptic agent delivery system 300 and a handle 322 for directing nozzle 310. The antiseptic agent delivery system 300 may include components that are similar to, or the same as, the antiseptic agent delivery system 100 of FIG. 1. For example, housing 302, conduit 308, and nozzle 310 are illustrated. It should be appreciated that antiseptic agent delivery system 300 may include other components such as an ultrasound generator, though they may not be shown.

For example, FIG. 4 may illustrate an example antiseptic agent delivery system, in accordance with various embodiments of the present disclosure. As illustrated, the antiseptic agent delivery system 400 may be portable. The antiseptic agent delivery system 400 may include a handle 430 for holding the antiseptic agent delivery system 400, and a trigger 432 for turning on the antiseptic agent delivery system 400 and related components thereto. The antiseptic agent delivery system 400 may include components that are similar to, or the same as, the antiseptic agent delivery system 100 of FIG. 1. For example, housing 402 including a cap 403, reservoir 404, conduit 408, nozzle 410, and power system 414 are illustrated. It should be appreciated that antiseptic agent delivery system 400 may include other components such as an ultrasound generator, though they may not be shown.

Referring back to FIG. 1, in some embodiments, the one or components housed within housing 102 may cause the aqueous antiseptic solution to be vaporized from the aqueous antiseptic solution. For example, housing 102 may include ultrasound generator 106 to transmit ultrasonic waves through the aqueous antiseptic solution. In some embodiments, one or more ultrasound generators may be used. In some embodiments, the housing 102 may include an inlet (not shown) through which air is supplied under pressure from a compressor (not shown) to drive the vapor out the nozzle 110. In some embodiments, antiseptic agent delivery system 100 may be configured to use a driving gas flow (e.g., typically 0.5 mL/min) to drive vapor out the nozzle 110. For example, the foggers may deliver an approximately equal volume of vapor during the inhalation phase (i.e., when patient is breathing). In some embodiments, the pressurized air may be directed via an air channel (not shown) into the liquid supply reservoir 104 to direct the vapor adjacent the solution surface through the nozzle 110.

In some embodiments, the antiseptic agent delivery system 100 may be configured to carry the vapor through a conduit 108 connected to the nozzle 110. In some embodiments, where the nozzle 110 is a mouthpiece, the patient may aspirate the vapor antiseptic solution through the nozzle 110. In some embodiments, the diameter of vapor may be approximately 1 to 5 microns to ensure the particles or droplets are not likely to be impacted in the airway before they reach the lungs and are not carried out of the lungs again on exhalation without being deposited within the respiratory system structures (e.g., lungs).

In some embodiments, the conduit 108 may be configured to be slightly larger in diameter than an exit port (not shown) within the nozzle 110. By virtue of the conduit 108 being slightly larger in diameter than the exit port of the nozzle 100, a small space between the outer surface of the air exit port and the inner surface of the conduit 108 is provided. For example, the space may be approximately 0.00254-0.254 mm. In some embodiments, the space allows fluid from the liquid supply reservoir 104 to proceed upward between the air exit port and the conduit 108. In some embodiments, the diameter of the conduit 108 may be adjusted to change the particle size of the vapor. It should be appreciated that the ultrasonic vibrations may be controlled with a control panel (not shown) to increase or decrease the particle size of the vapor. There may also be a control on the control panel (not shown) to increase the rate of vapor coming out of the nozzle 110 by affecting the compressor, driving gas flow, or other active mechanism to drive the vapor out the nozzle 110.

In some embodiments, the housing 102 may include one or more sensors (not shown) configured to detect the pressure within the liquid supply reservoir 104 and/or the vibrations of the ultrasound generator 106. In some embodiments, the one or more sensors may be connected to the inside of the nozzle 110, liquid supply reservoir 104, or adjacent to the ultrasound generator 106. In some embodiments, the one or more sensors may detect how much volume has been distributed by the antiseptic agent delivery system 100, the vibration level of the ultrasound generator 106, and/or other information.

In one example, the one or more sensors may detect that a patient has inhaled causing the antiseptic agent delivery system 100 to divert pressurized air to an air outlet (not shown). In some embodiments, the antiseptic agent delivery system 100 may be configured to analyze the pressure changes within the system 100 during a certain number of initial breaths (e.g., first three breaths) to determine an average shape of the breathing pattern. A timed pulse of vaporization may be commenced upon start of subsequent inspirations such that vaporization occurs for the first 50 percent of the inspiration. In some embodiments, the antiseptic agent delivery system 100 may be configured to have a timed pulse of vaporization to occur during a period other than 50% of the duration of inspiration. In some embodiments, the antiseptic agent delivery system 100 may be configured to have a predetermined pulse length. For example, the pulse length may be set for each patient by a clinician.

In some embodiments, the housing 102 may include one or more panels (not shown) to operate the one or more components configured to vaporize the aqueous antiseptic solution. For example, a timing mechanism may allow the antiseptic agent delivery system 100 to be run for a given number of minutes (e.g., 5 minutes, 1 hour, 6 hours, 12 hours, 24 hours, and so on in various increments of time). In some embodiments, the housing 102 may include a power system 114 to which a power cable (not shown) may be connected. In some embodiments, the power system 114 may be one or more batteries.

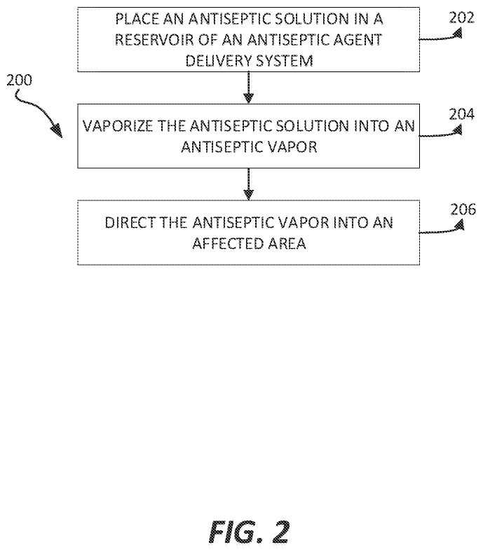

In some embodiments, microbes (including spores), viruses, fungi, allergy-causing agents, and other sources of infectious diseases may be killed, disinfected, or otherwise made ineffective by administering, distributing, delivering, and/or otherwise applying HOCl to the affected area. As an exemplary list, the infectious disease and/or their sources may include diseases caused by microbes (including spores), antimicrobes, pollutants, microorganisms, biofilms, viruses, bacteria, fungi, protists, parasites, allergy-causing agents, and/or other organisms, including mast cell degranulation, acne, pneumonia, biofilms, bronchiectasis, asthma, acute respiratory distress syndrome (ARDS), bronchitis, sleep apnea, chronic obstructive pulmonary disease (COPD), chest infections, cystic fibrosis, tuberculosis, liver cirrhosis, staphlococcus aureus, haemophilius influenzae, klebsiella pnuemoniae, pseudomona aeruginosa, bordetella pertussis, moraxella catarrhalis, coxiella burnetiid, chlamdyophilia pneumoniae, mycoplasma pneumoniae, legionella pneumophilia, yesinia pestis, influenza viruses, rhinoviruses, respiratory syncytial virus, adenovirus, enterovirus, parainfluenza, Epstein-Barr virus, cytomegalovirus, hantavirus, Herpes simplex, histoplasma capsulatum, blastomyces, pneumocystis, coccidiodes, thrush, herpes simplex ulcers, other infections of the mouth, otitis media, cavity-causing bacteria, gingivitis, Helicobacter pylori, Giardia, tapeworms, Entamoeba, other GI-infecting organisms, Clostridium difficile, colitis, diarrhea, Candida, vaginitis, drug-resistant bacteria, pruritic, and the like. Embodiments using this method may involve administering, distributing, delivering, and/or otherwise applying HOCl to an affected area using the antiseptic agent delivery system. For example, and as illustrated in FIG. 2, the administration, distribution, delivery, and/or application of the vaporized HOCl may include one or more of the following operations. In an operation 202, an aqueous antiseptic solution may be placed in a reservoir of the antiseptic agent delivery system. For example, the aqueous antiseptic solution comprising HOCl ranging in volume from approximately 0.5 ml to 10 ml may be placed into the reservoir. In an operation 204, the aqueous antiseptic solution may be vaporized into an antiseptic vapor. For example, the aqueous antiseptic solution comprising HOCl may be vaporized into particles ranging from approximately 0.1 μm to 99 μm or larger, in size. In an operation 206, the aqueous antiseptic vapor may be directed into an affected area. For example, the vaporized aqueous antiseptic solution comprising HOCl may be aspired for a prescribed duration (e.g., a period ranging from approximately 0.5 min to 30 min. or longer).

In some embodiments, illness suspected to be caused by microbial (including spores), viral, fungal, allergy-causing agents may be treated by administering, distributing, delivering, and/or otherwise applying HOCl through use of a fogger. For example, a fogger may be used to transform the aqueous solution of HOCl into a vapor for administering, distributing, delivering, and/or otherwise applying the solution to the oral cavity and/or throat structures (e.g., oropharynx, larynx, etc.). In embodiments, oral inflammation and/or ulceration (e.g., mucositis) which may arise as an adverse effect to a particular medication (e.g., chemotherapy and radiotherapy treatment for cancer) or due to dehydration, poor mouth care, oxygen therapy, excessive use of alcohol and/or tobacco, and lack of protein in the diet may be treated using the HOCl vapor. In some embodiments, the aqueous solution of HOCl may be diluted with one or more diluents. For example, approximately 0.5 ml to 20 ml of saline may be added to 10 ml of the aqueous solution of HOCl. It should be appreciated that the diluted HOCl may be used in any of the embodiments discussed herein.

In some embodiments, one or more effects of relaxing one or more respiratory structures (e.g., uvula, soft palate, etc.) resulting in a sound (e.g., snoring) due to their vibrations during sleep may be treated by administering, distributing, delivering, and/or otherwise applying HOCl. For example, the HOCl solution may be administered, distributed, delivered, and/or otherwise applied by a pulmonary delivery method or nasal delivery method.

In some embodiments, an irritation, inflammation, and/or obstruction of the breathing passages resulting in a cough reflex and often associated with acute and/or chronic respiratory tract infection may be treated by administering, distributing, delivering, and/or otherwise applying HOCl.

In some embodiments, an irritation and/or an inflammation of the voice box resulting in loss of voice and/or diminished capacity to produce sound (e.g., laryngitis) may be treated by administering, distributing, delivering, and/or otherwise applying HOCl.

In some embodiments, an irritation and/or an inflammation of one or more structures within the nasal cavity and/or throat due to an allergic reaction to one or more allergens, such as pet dander, dust, mites, pollen and mold, may be treated by administering, distributing, delivering, and/or otherwise applying HOCl. For example, the HOCl solution may be administered, distributed, delivered, and/or otherwise applied by a pulmonary delivery method, as described above. Alternatively, the HOCl solution may be used to decrease the histamine response which may be elevated during an allergic response to one or more allergens, as previously alluded to.

In some embodiments, the pH level of the HOCl solution administered, distributed, delivered, and/or otherwise applied through the methods disclosed herein may be pH-neutral because stabilized and/or pH-neutral HOCl is superior in terms of antimicrobial activity to non-stabilized HOCl and acidified bleach, including against hypochlorite-resistant strains. In some embodiments, the acidotic pH level of the HOCl may be within the range resulting in the highest amount of undissolved HOCl. For example, the acidotic pH level may range from approximately pH 4 to approximately pH 7.

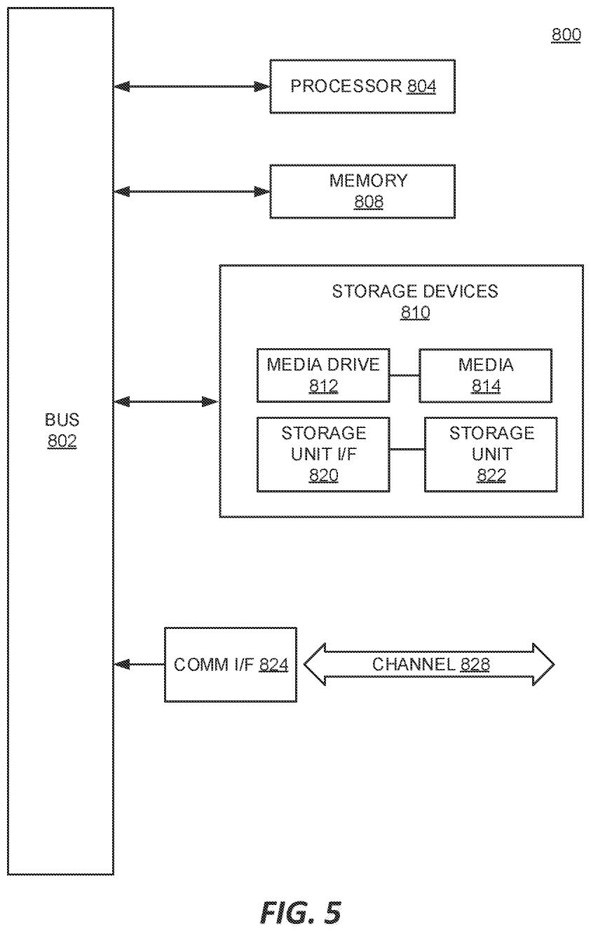

As used herein, the term component might describe a given unit of functionality that can be performed in accordance with one or more embodiments of the technology disclosed herein. As used herein, a component might be implemented utilizing any form of hardware, software, or a combination thereof. For example, one or more processors, controllers, ASICs, PLAs, PALs, CPLDs, FPGAs, logical components, software routines or other mechanisms might be implemented to make up a component. In implementation, the various components described herein might be implemented as discrete components or the functions and features described can be shared in part or in total among one or more components. In other words, as would be apparent to one of ordinary skill in the art after reading this description, the various features and functionality described herein may be implemented in any given application and can be implemented in one or more separate or shared components in various combinations and permutations. As used herein, the term engine may describe a collection of components configured to perform one or more specific tasks. Even though various features or elements of functionality may be individually described or claimed as separate components or engines, one of ordinary skill in the art will understand that these features and functionality can be shared among one or more common software and hardware elements, and such description shall not require or imply that separate hardware or software components are used to implement such features or functionality.

Where engines and/or components of the technology are implemented in whole or in part using software, in one embodiment, these software elements can be implemented to operate with a computing or processing component capable of carrying out the functionality described with respect thereto. One such example computing component is shown in FIG. 5. Various embodiments are described in terms of this example-computing component 800. After reading this description, it should be appreciated how to implement the technology using other computing components or architectures.

Referring now to FIG. 5, computing component 800 may represent, for example, computing or processing capabilities found within desktop, laptop, and notebook computers; hand-held computing devices (PDA's, smart phones, cell phones, palmtops, etc.); mainframes, supercomputers, workstations, or servers; or any other type of special-purpose or general-purpose computing devices as may be desirable or appropriate for a given application or environment. Computing component 800 might also represent computing capabilities embedded within or otherwise available to a given device. For example, a computing component might be found in other electronic devices such as, for example, digital cameras, navigation systems, cellular telephones, portable computing devices, modems, routers, WAPs, terminals and other electronic devices that might include some form of processing capability.

Computing component 800 might include, for example, one or more processors, controllers, control components, or other processing devices, such as a processor 804. Processor 804 might be implemented using a general-purpose or special-purpose processing engine such as, for example, a physical computer processor, microprocessor, controller, or other control logic. In the illustrated example, processor 804 is connected to a bus 802, although any communication medium can be used to facilitate interaction with other components of computing component 800 or to communicate externally.

Computing component 800 might also include one or more memory components, simply referred to herein as main memory 808. For example, random access memory (RAM) or other dynamic memory might be used for storing information and instructions to be executed by processor 804. Main memory 808 might also be used for storing temporary variables or other intermediate information during execution of instructions to be executed by processor 804. Computing component 800 might likewise include a read-only memory (“ROM”) or other static storage device coupled to bus 802 for storing static information and instructions for processor 804.

The computing component 800 might also include one or more various forms of information storage device 810, which might include, for example, a media drive 812 and a storage unit interface 820. The media drive 812 might include a drive or other mechanism to support fixed or removable storage media 814. For example, a hard disk drive, a floppy disk drive, a magnetic tape drive, an optical disk drive, a CD or DVD drive (R or RW), or other removable or fixed media drive might be provided. Accordingly, storage media 814 might include, for example, non-transient electronic storage, a hard disk, a floppy disk, magnetic tape, cartridge, optical disk, a CD or DVD, or other fixed or removable medium that is read by, written to, or accessed by media drive 812. As these examples illustrate, the storage media 814 can include a computer usable storage medium having stored therein computer software or data.

In alternative embodiments, information storage mechanism 810 might include other similar instrumentalities for allowing computer programs or other instructions or data to be loaded into computing component 800. Such instrumentalities might include, for example, a fixed or removable storage unit 822 and an interface 820. Examples of such storage units 822 and interfaces 820 can include a program cartridge and cartridge interface, a removable memory (for example, a flash memory or other removable memory component) and memory slot, a PCMCIA slot and card, and other fixed or removable storage units 822 and interfaces 820 that allow software and data to be transferred from the storage unit 822 to computing component 800.

Computing component 800 might also include a communications interface 824. Communications interface 824 might be used to allow software and data to be transferred between computing component 800 and external devices. Examples of communications interface 824 might include a modem or softmodem, a network interface (such as an Ethernet, network interface card, WiMedia, IEEE 802.XX, or other interface), a communications port (such as for example, a USB port, IR port, RS232 port, Bluetooth® interface, or other port), or other communications interface. Software and data transferred via communications interface 824 might typically be carried on signals, which can be electronic, electromagnetic (which includes optical), or other signals capable of being exchanged by a given communications interface 824. These signals might be provided to communications interface 824 via channel 828. This channel 828 might carry signals and might be implemented using a wired or wireless communication medium. Some examples of a channel might include a phone line, a cellular link, an RF link, an optical link, a network interface, a local or wide area network, and other wired or wireless communications channels.

In this document, the terms “computer program medium” and “computer usable medium” are used to generally refer to media such as, for example, memory 808, storage unit 820, media 814, and channel 828. These and other various forms of computer program media or computer usable media may be involved in carrying one or more sequences of one or more instructions to a processing device for execution. Such instructions embodied on the medium are generally referred to as “computer program code” or a “computer program product” (which may be grouped in the form of computer programs or other groupings). When executed, such instructions might enable the computing component 800 to perform features or functions of the disclosed technology as discussed herein.

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that can be included in the disclosed technology. The disclosed technology is not restricted to the illustrated example architectures or configurations, but the desired features can be implemented using a variety of alternative architectures and configurations. Indeed, it will be apparent to one of skill in the art how alternative functional, logical or physical partitioning, and configurations can be implemented to implement the desired features of the technology disclosed herein. Also, a multitude of different constituent component names other than those depicted herein can be applied to the various partitions. Additionally, with regard to flow diagrams, operational descriptions, and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects, and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known,” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to,” or other like phrases in some instances shall not be read to mean that the narrower case is intended or required in instances where such broadening phrases may be absent. The use of the term “component” does not imply that the components or functionality described or claimed as part of the component are all configured in a common package. Indeed, any or all of the various components of a component, whether control logic or other components, can be combined in a single package or separately maintained and can further be distributed in multiple groupings or packages or across multiple locations.

Additionally, the various embodiments set forth herein are described in terms of exemplary block diagrams, flow charts, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. An antiseptic agent delivery system comprising:
a reservoir configured to store an antiseptic solution, wherein the antiseptic solution comprises an aqueous solution of hypochlorous acid;
an ultrasound generator coupled to the reservoir, wherein the ultrasound generator vaporizes the antiseptic solution into an antiseptic vapor;
a nozzle to direct the antiseptic vapor to an affected area; and
a housing coupled to the reservoir and the nozzle, wherein the housing comprises a panel to control the ultrasound generator.

2. The system of claim 1, wherein the hypochlorous acid has a pH range of approximately 4 to 7.

3. The system of claim 1, wherein the affected area comprises one of a bacteria, a virus, a yeast, a mold, a fungus, a spore, a protozoa, and a prion.

4. The system of claim 1, wherein a size of the antiseptic vapor ranges from approximately 0.5 μm to approximately 50 μm.

5. The system of claim 1, wherein the antiseptic agent delivery system further comprises a mechanism coupled to the reservoir to push the antiseptic vapor from the reservoir out the nozzle.

6. The system of claim 1, wherein the ultrasound generator comprises one of a transducer, oscillator, and a compressor.

7. The system of claim 1, wherein the ultrasound generator comprises an electronic oscillator and one or more piezoelectric elements.

8. A method of disinfecting an affected area, the method comprising:
delivering an antiseptic solution to the affected area using an antiseptic agent delivery system, wherein the antiseptic agent delivery system comprises:
a reservoir configured to store an antiseptic solution, wherein the antiseptic solution comprises an aqueous solution of hypochlorous acid;
an ultrasound generator coupled to the reservoir, wherein the ultrasound generator vaporizes the antiseptic solution into an antiseptic vapor;
a nozzle to direct the antiseptic vapor to an affected area; and
a housing coupled to the reservoir and the nozzle, wherein the housing comprises a panel to control the ultrasound generator; and
wherein the antiseptic solution comprises an aqueous solution of hypochlorous acid.

9. The method of claim 8, wherein the distribution of the antiseptic solution comprises:
placing the antiseptic solution in the reservoir;
transforming the antiseptic solution into the antiseptic vapor using the ultrasound generator; and
directing the antiseptic vapor to the affected area using the nozzle.

10. The method of claim 8, wherein the hypochlorous acid has a pH range of approximately 4 to 7.

11. The method of claim 8, wherein the affected area comprises one of a bacteria, a virus, a yeast, a mold, a fungus, a spore, a protozoa, and a prion.

12. The method of claim 8, wherein a size of the antiseptic vapor ranges from approximately 0.5 μm to approximately 50 μm.

13. The method of claim 8, wherein the ultrasound generator comprises one of a transducer, oscillator, and a compressor.

14. The method of claim 8, wherein the ultrasound generator comprises an electronic oscillator and one or more piezoelectric elements.

15. A method of distributing an antiseptic solution, the method comprising:
distributing the antiseptic solution using an antiseptic agent delivery system, wherein the antiseptic agent delivery system comprises:
a reservoir configured to store an antiseptic solution, wherein the antiseptic solution comprises an aqueous solution of hypochlorous acid;
an ultrasound generator coupled to the reservoir, wherein the ultrasound generator vaporizes the antiseptic solution into an antiseptic vapor;
a nozzle to direct the antiseptic vapor to an affected area; and
a housing coupled to the reservoir and the nozzle, wherein the housing comprises a panel to control the ultrasound generator; and
wherein the antiseptic solution comprises an aqueous solution of hypochlorous acid.

16. The method of claim 15, wherein the hypochlorous acid has a pH range of approximately 4 to 7.

17. The method of claim 8, wherein the affected area comprises one of a bacteria, a virus, a yeast, a mold, a fungus, a spore, a protozoa, and a prion.

18. The method of claim 15, wherein a size of the antiseptic vapor ranges from approximately 0.5 μm to approximately 50 μm.

19. The method of claim 15, wherein the hypochlorous acid is between 30 parts per million (ppm) to 500 ppm of the antiseptic solution.

20. The method of claim 15, wherein the ultrasound generator comprises an electronic oscillator and one or more piezoelectric elements.
